SEQUENCE LISTING (5 sequences)

SEQ ID NO: 1 - RNA, 17 nt, Homo sapiens
gcggggcugggcgcgcg

SEQ ID NO: 2 - RNA, 18 nt, Homo sapiens
ugaggcgggggggcgagc

SEQ ID NO: 3 - RNA, 22 nt, Homo sapiens
uuugguccccuucaaccagcug

SEQ ID NO: 4 - RNA, 22 nt, Homo sapiens
uggaauguaaagaaguauguau

SEQ ID NO: 5 - RNA, 17 nt, Homo sapiens
ggggcugggcgcgcgcc

(19) United States
(12) Patent Application Publication
(10) Pub. No.: US 2019/0284631 A1
(43) Pub. Date: Sep. 19, 2019

(54) Personalized Healthcare P4 Alzheimer's Detection System and Method

(71) Applicant: eNano Health Limited, Hong Kong SAR (HK)

(72) Inventor: Patrick Shau-park Leung, Arcadia, CA (US)

(73) Assignee: eNano Health Limited, Hong Kong SAR (HK)

(21) Appl. No.: 16/374,838

(22) Filed: Apr. 4, 2019

Related U.S. Application Data
(63) Continuation-in-part of application No. 15/056,163, filed on Feb. 29, 2016.
Application No. 15/056,163 is a continuation-in-part of application No. 15/469,138, filed on Mar. 24, 2017.
(60) Provisional application No. 62/653,540, filed on Apr. 5, 2018.

(51) Int. Cl. C12Q 1/6883; G01N 33/487; G16B 40/00; G16H 50/30

(52) U.S. Cl. CPC C12Q 1/6883; G01N 33/4875; G01N 33/48792; C12Q 2600/158; G16H 50/30; C12Q 2600/118; G16B 40/00

(57) ABSTRACT

The claimed invention provides real-time and subsequent analysis personalized user based health and wellness information for predictive Alzheimer's diagnosis information. Non-invasive techniques utilize saliva for body levels of wellness indicators and microRNA predictive markers which are coordinated over time. Saliva captured on lateral flow sample collection strips are real-time indicator reviewed and subsequently analyzed using optional traditional analytical chemistry techniques including liquid chromatography/mass spectrometry (LC/MS) and coordinated with time of administration with genetic sequence analysis to confirm related disease conditions. By using P4 (Participatory, Personalized, Predictive, and Preventive) health management techniques the patient determines if telltale correlative microRNA indicators are present.

### CITATION LIST

### Patent Literature

This patent application claims priority to provisional patent application 62/653,540 filed Apr. 5, 2018. Furthermore this patent application is a continuation-in-part and claims priority to U.S. patent application Ser. No. 15/666,699 filed Aug. 2, 2017 to Patrick Shau-park Leung entitled “Personalized Glucose and Insulin Monitoring System.” In addition, this patent application is a continuation-in-part and claims priority to U.S. patent application Ser. No. 15/469,138 filed Mar. 24, 2017 to Patrick Shau-park Leung entitled “Public personalized mobile health sensing system, method and device” which is a continuation of U.S. patent application Ser. No. 15/056,163 filed Feb. 29, 2016 to Patrick Shau-park Leung entitled “Mobile automated health sensing system, method and device”.

### TECHNICAL FIELD

The claimed invention relates to biomedical healthcare patient monitoring based upon the P4 (Participatory, Personalized, Predictive, and Preventive) health management method. With greater particularity, the claimed invention addresses personalized monitoring of Alzheimer's disease conditions with patient alerting and artificial intelligence data interpretation.

### BACKGROUND ART

Traditional biomedical monitoring of patient pharmaceutical administration is often clinical in nature with results ordered by a doctor in a hospital or medical office setting and performed in a centralized laboratory setting. Even when patients are informed as to the blood levels of their pharmaceutical body chemistry it is often through the lens of the primary medical provider.

Using traditional methods, if a patient wishes to know detailed information about personal pharmaceutical levels in the body they must first schedule an office visit. Absent an emergency, such visits usually take place weeks to months after the request is made. To determine body levels of pharmaceutical products ingested, blood is drawn and sent to an outside laboratory. Several days later the results are reported back to the primary healthcare physician who interprets the laboratory results and provides a high level summary to the patient. Despite the rapid expansion of ‘big data’ healthcare information, patients are rarely the owners or curators of their own healthcare information leading to reduced choices and far fewer options in healthcare data portability when seeking out alternate providers.

Alzheimer's in particular has proven difficult to diagnose and generally results in patient information ‘silos’ which prevent a full wellness picture to enable greater patient healthcare options.

### SUMMARY OF INVENTION

### Technical Problem

Current systems for Alzheimer's patient diagnosis are centralized and exclusionary. They are not participatory apart from the clinical samples that the patient provides for testing. Reporting of diagnostic results are not personalized in that apart from the unique data itself released by a medical healthcare provider, the medical service provider controls the manner, method and timing of information content release. The technical problems of early identification of an Alzheimer's diagnosis are primarily systematic in nature due to legal and healthcare provider process constraints around the information itself

New models of Alzheimer's early detection are rapidly developing but patient access often lags far behind owing to delays in medical education and practitioner adoption. In addition, traditional laboratory nitrocellulose paper is often unsuitable for sample collection conjugated with analytical reporting chemicals.

### Solution to Problem

By embracing the P4 (Participatory, Personalized, Predictive, and Preventive) health management method, the claimed invention provides patient engaging Alzheimer's indication information. By utilizing patient saliva samples which are locally analyzed then transported to a centralized analysis facility, information relevant to early Alzheimer's indications are accurately captured and rapidly delivered to the patient and healthcare providers using a smartphone or personal computing device.

Patient glucose level information is non-invasively obtained by saliva samples collected on disposable sample means including lateral flow sample collection strips. Local real-time analysis is complemented by subsequent transportation to a centralized analytical facility using traditional laboratory equipment including Liquid Chromatography/Mass Spectrometry (LC/MS) including protein analysis, Elisa chemical analysis as well as next generation sequencing of micro-RNA (miRNA) and DNA.

While competing models of Alzheimer's risk factors undergo further analysis, patients can actively monitor glucose wellness indicators in real time while tracking potential risk factors over time. Samples taken from saliva specimens captured during glucose monitoring are stable at room temperature and can be reliably transported to centralized analytical facilities. Potential Alzheimer's indicators screened using traditional laboratory equipment include differential analysis of multiple microRNA including miR-4508, miR-6087, miR-133a-3p, miR-1-3-p and miR-4492. Complementary indicators from telltale fungal, viral and microbial risk factors are also weighted and assessed. Owing to the stability of the saliva samples, representative source lateral flow sample collection strips can be archived and subsequently retested as new risk factors are identified. In addition, enhancements to salivary sample capture in combination with analytical reporting chemicals include optimized lateral flow strip material.

### Advantageous Effects of Invention

By empowering the patient to cultivate their own Alzheimer's risk factor information, predictive and preventative wellness is enabled. Early identification of Alzheimer's allows for early adoption of non-invasive cognitive therapy techniques for maximum therapeutic benefits. More importantly, the claimed invention utilizing recently characterized microRNA which are novel as indicators for Alzheimer's provide an early assessment tool rapidly identifying risk factors not identified by traditional diagnostic kits presently on the market.

In addition to glucose monitoring enabled behavioral changes, the claimed invention enables direct monitoring for and analysis of telltale microRNA indicators present in Alzheimer's which are correspondingly absent in healthy individuals. The microRNA analysis may be conducted independently from and in the absence of real-time glucose analysis or may be complementary to patient glucose analysis. Current models for Alzheimer's are targeting fungal, viral and microbial sources of Alzheimer's either as a disease source or telltale indicator. By analyzing patient saliva samples for telltale microRNA as well as fungal, viral and bacterial risk factors identified using next generation sequencing, potential risk factors can be identified early and mitigated sooner allowing for the potential for Alzheimer's disease mitigation or potential avoidance.

In a doctor's office, an Alzheimer's patient consultation reflects a single point of time measured infrequently separated by months or years. In the claimed invention, with regular patient monitoring it is an expected and intended consequence that a deeper and more personalized wellness profile is generated by regularly tracking salivary glucose levels complemented by or alternatively independently monitoring of telltale microRNA indicators as well as fungal, viral and bacterial Alzheimer's risk factors.

### BRIEF DESCRIPTION OF DRAWINGS

The accompanying drawings are included to better illustrate exemplary embodiments of the claimed invention.

FIG. 1 is a schematic illustration of Alzheimer's disease threat factors and microRNA indicators.

FIG. 2 is a top level schematic illustration of saliva lateral flow sample collection strip.

FIG. 3 is a side view schematic illustration of saliva lateral flow sample collection strip with enhanced functionality.

FIG. 4 is a flowchart illustrating a preferred embodiment of the claimed invention.

FIG. 5 is a flowchart illustrating a preferred embodiment of the claimed invention.

### DESCRIPTION OF EMBODIMENTS

P4 Medicine is Predictive, Preventive, Personalized and Participatory. Its two major objectives are to quantify wellness and demystify disease. In the illustrative examples contained herein, the aims of P4 Medicine are achieved by combining end-user analysis of current health metrics together with follow-on lab analytics of the same saliva sample to determine body levels microRNA with prognostic Alzheimer's indications.

Optionally, the system may be combined with glucose measuring test strips to report glucose levels to the end-user for personalized and participatory wellness monitoring. The same test strip subsequently analyzed using standard analytical equipment, however, provides the opportunity for predictive and preventative health screening based upon detection of pharmaceuticals and their carriers as well as DNA, RNA and protein indicators of body health as well as the presence or absence of harmful bacteria, viruses and other disease carriers.

### EXAMPLES

### Example 1

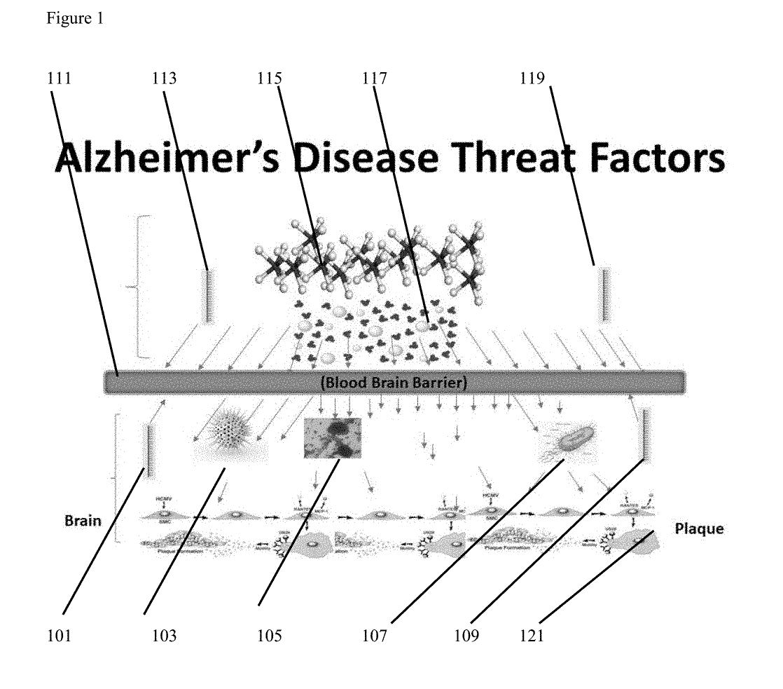

The claimed P4 Alzheimer's wellness platform is based upon salivary capture and analysis using one or more disposable lateral flow sample collection test strips. FIG. 1 depicts an illustrative schematic model of Alzheimer's threat indicators as reflected by microRNA (101, 109, 113, 119) present on both sides of the blood brain barrier (111) as well as fungal (105), bacterial (107) and viral (103) threats leading to the creation of plaque (121). Test strips (not shown) capture saliva based biomarkers capable of passing the blood brain barrier (111) including small molecules (117) and impermeable large molecules (115). Examples of Alzheimer's risk factor microRNA include microRNA 4508, microRNA-6087, microRNA-133a-3p, microRNA-1-3p and microRNA-4492.

The claimed invention is distinguishable from traditional views of neurological disease such as Alzheimer's disease. Rather than a single correlative ‘one to one’ microRNA to disease state model, the claimed invention utilizes differential analysis of a panel of microRNA present in saliva to indicate potential for onset of Alzheimer's disease. In a preferred embodiment, miR-4508, miR-1-3p, miR-133a-3p, miR-4492 and miR-6087 are used for detecting Alzheimer's disease as reflected in Table 1.

TABLE 1
Alzheimer's Panel Screening MicroRNA Description

| MicroRNA | Sequence |
|---|---|
| miR-4508 | GCGGGGCUGGGCGCGCG |
| mir-6087 | UGAGGCGGGGGGGCGAGC |
| mir-133-3p | UUUGGUCCCCUUCAACCAGCUG |
| Mir-1-3p | uggaauguaaagaaguauguau |
| mir-4492 | GGGGCUGGGCGCGCGCC |

The Alzheimer's predictive miR-4508, miR-1-3p, miR-133a-3p, miR-4492 and miR-6087 are not normally found in the saliva of healthy individuals but are present in Alzheimer's patients as reflected in Table 2. In particular, miRNA-4508 and 4492 are not present in the exosome of normal neural stem cells while are present in the exosome of abnormal neural stem cells.

TABLE 2
Alzheimer's Panel Screening MicroRNA Indicative Levels

| | | | | Normalized |
|---|---|---|---|---|
| | | | | Disease/ |
| | AZ set 1 | AZ set 2 | Control | Control |
| MicroRNA | Normalized | Normalized | Normalized | Ratio |
| miR-4508 | 147 | 28 | 0 | 99999 |
| mir-6087 | 812 | 60 | 0 | 99999 |
| mir-133-3p | 239 | 53 | 0 | 99999 |
| Mir-1-3p | 582 | 338 | 0 | 99999 |
| mir-4492 | 1229 | 214 | 0 | 99999 |

Based on differential analysis of microRNA levels of Table 2 prognostic indicators present or absent in saliva samples analyzed by genetic sequencing, risk factors alerting to the onset of Alzheimer's are reported according to the claimed invention as demonstrated in the illustrative examples.

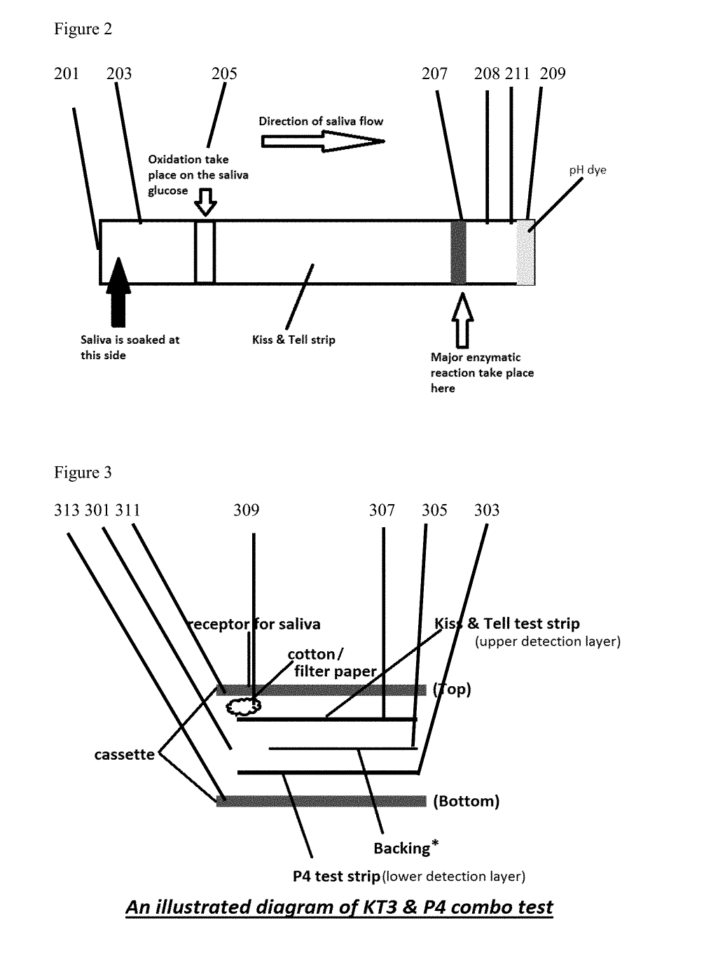

FIG. 2 depicts salivary test strip (201) which captures saliva (not shown) at salivary capture area (203) which is distributed by lateral flow into oxidation region (205) and onto enzymatic region (207) concluding with optional pH region (209). In the first illustrative embodiment the local enzymatic analysis provides locally measurable salivary indicator levels and may additionally incorporate antibody indicator region (208) as well as optional aptamer indicator region (211).

In the first illustrative example, Alzheimer's prognosticative microRNA levels are captured by placing test strip (201) in a user's mouth (not shown) for two minutes to distribute saliva (not shown) to test strip (201). Adequate saliva capture is confirmed by illumination of pH region (209). In the first illustrative example, the user waits an additional three minutes upon which a measurable color change takes place at enzymatic region (207). The complementary detection of salivary glucose is based on a coupling reaction between glucose oxidase and peroxidase. Glucose oxidase oxidizes the salivary glucose into gluconolactone and hydrogen peroxide (H2O2). In the presence of peroxidase, 10-acetyl-3,7-dihydroxyphenoxazine reacts with H2O2 in a 1:1 stoichiometry in order to produce a white to pink color. In a preferred embodiment, the chemical sensor at enzymatic region (207) is a compound having the following structural formula:

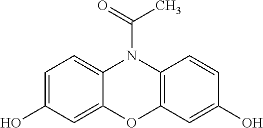 [SMILES: CC(=O)N1c2ccc(O)cc2Oc2cc(O)ccc21]

Salivary indicator levels may be estimated by user color comparison visually or by computer analysis by a smartphone type device (not shown).

In the first illustrative embodiment, the salivary test strip may be single layer as illustrated by salivary test strip (201) depicted by FIG. 2 or multi-layer as illustrated by multi-function salivary test strip (301) depicted in FIG. 3. FIG. 3 multi-function salivary test strip (301) is multi-layer with top analytical layer (307), layer divider (305) backing and lower analytical layer (303). Saliva access is provided through optional cassette housing (313) with salivary receptacle (311) which distributes saliva (not shown) through optional saliva wicking material (309) which can be cotton, filter paper or other material suitable for distribution of saliva. In a preferred embodiment, optimized analytical lateral flow material is utilized for top analytical layer (307) and/or lower analytical layer (303) which is distinguishable from traditional nitrocellulose filter paper by absorbency rate and internal composition. Distinguishable characteristics from traditional nitrocellulose paper include high hydrophilic behavior wicking 4 cm in under 50 seconds. Optimal analytical flow material characteristics include highly efficient body fluid separation with no analyte interference, excellent release with both latex and gold conjugates, reaction membrane to capture reagents bound to the immobilized latex beads combined with conjugate and analyte to give intense capture lines and superior sample wicking with no loss of assay sensitivity when compared to other materials and acting as an absorbent to liquids.

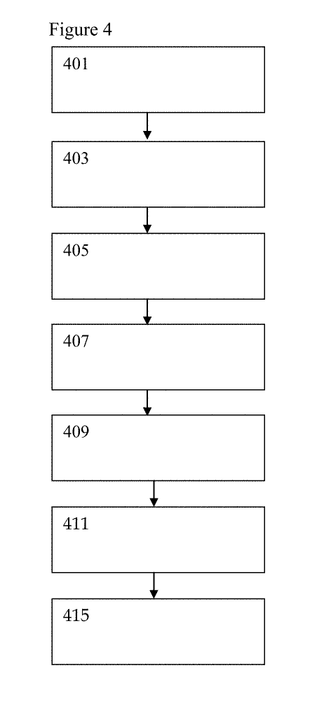

FIG. 4 illustrates the process of utilizing the claimed invention to assess and monitor Alzheimer's risk factors. Sample preparation step (401) begins with the user placing saliva on a sample collection means and the system stores the time of saliva sample capture. In the illustrative embodiment the saliva sample is captured by the user on a lateral flow sample collection strip which may be enhanced with an optional glucose level indicator as further illustrated in the second illustrative embodiment. Optional glucose data capture step (403) is achieved by a patient capturing glucose levels in real-time by smartphone camera. The sample is sent by mail or otherwise transported to a central analysis facility and optionally analyzed by liquid chromatography and mass spectrometry (LC/MS) in addition to sample genetic analysis step (405) to determine body levels of microRNA indicative of Alzheimer's. While the illustrative example utilizes a centralized genetic analysis platform screening for fungal, viral and bacterial contributing risk factors complemented by LC/MS and ELIZA other foreseen and intended variants may utilize localized dedicated analysis platforms.

The remainder of the first illustrative embodiment illustrated by FIG. 4 takes place in a computational or cloud computing environment. During data analysis step (407) body levels of microRNA risk factors are assessed together with body glucose levels. Data transmission step (409) transmits the user results to the user's preferred computational device including smartphone and smart watch. Data reporting step (411) provides the user with microRNA Alzheimer's risk factor levels. Optional data alert/feedback gathering step (413) reports abnormal or medically dangerous risk factor levels to the user as well as medical providers and designated family members and provides an opportunity for gathering user feedback. Data mining step (415) provides a deeper analysis into Alzheimer's risk factor levels as a function of time and behavior as greater data is collected by the system.

### Example 2

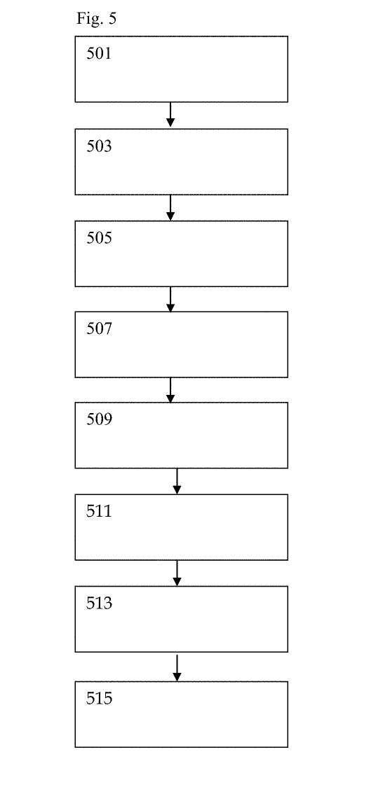

In a second illustrative example, expanded Alzheimer's personalized wellness information is obtained by augmenting real-time glucose sensing with subsequent LC/MS and ELIZA analysis in conjunction with DNA and RNA sequencing of the saliva sample. In FIG. 5, sample preparation step (501) begins with a user in need of Alzheimer's monitoring placing a saliva sample collection means in the mouth to collect saliva and taking a digital photo of the lateral flow sample collection strip with a smartphone. The strip contains one or more glucose detection chemicals embedded in the saliva collection device which undergoes an optical or machine readable detection in real-time upon hybridization. After exposure to saliva the user takes a photo of the strip which captures the time of strip exposure and provides capture time and glucose level data to the system. The saliva capture means can be associated to the system by way of 2D bar code, machine readable numbers or other identifiable characteristics. Optional pharmaceutical data capture step (503) takes place with the user inputting pharmaceutical details of relevant pharmaceutical dosage and latest time of administration. Input may be through smartphone, smart watch or other dedicated computing device but by nature of the claimed invention is consumer user facing rather than and distinguishable from traditional lab bench analytical chemistry. After saliva exposure and smartphone photo capture the sample is placed into a prepaid envelope provided during purchase in the consumer packaging and is sent by mail or otherwise transported to a central analysis facility and analyzed by liquid chromatography and mass spectrometry (LC/MS) as well as genetic sequencing during sample chemical and genetic analysis step (505). Unlike blood or other biological material collection, the saliva sample is safe at room temperature and does not create hazardous waste handling concerns.

Data analysis step (507) takes place in a cloud computing environment to analyze glucose levels and genetic sequencing indicated microRNA telltale indicators. The Alzheimer's predictive miR-4508, miR-1-3p, miR-133a-3p, miR-4492 and miR-6087 are not normally found in the saliva of healthy individuals but are present in Alzheimer's patients as previously detailed in Table 2. In a foreseeable and intended embodiment the presence or absence of pharmaceutical carriers as well as multi-drug detection is carried out by the LC/MS system to determine if the pharmaceutical product is counterfeit and if the user is at risk from multi-drug cross reactions. In an intended alternate embodiment the presence or absence of illicit substances is also detected. Furthermore, the genetic sequencing and data analysis of the saliva sample allows for detection of fungal, bacterial and viral infections by screening for miRNA and DNA targets of interest.

The results are wirelessly transmitted over the internet during data transmission step (509) and the user's smartphone or smartwatch user interface displays a high level Alzheimer's risk factor metadata analysis during data reporting step (511).

Use of the claimed system is an iterative process, the more times the user provides results the more powerful the data becomes for user Alzheimer's wellness risk factor management. Optional data alert/feedback gathering step (513) is available to alert the user, designated family members and medical providers if critical microRNA threshold levels are breached. Feedback can also be obtained as a result of change in behavior and can be as simple as the system reporting ‘microRNA levels decreasing as a result of lifestyle changes, good work!” Data mining step (515) provides a deeper analysis into Alzheimer's microRNA levels as a function of time and behavior as greater data is collected by the system. While artificial intelligence cloud computing provides a computationally powerful tool, the smartphone/smart watch user interface report of data aggregation is intended to be simple by design. Aggregate results in this illustrative example are provided in a simple format for improved user personalized health.

In the description, numerous specific details are set forth in order to provide a thorough understanding of the present embodiments. It will be apparent, however, to one having ordinary skill in the art that the specific detail need not be employed to practice the present embodiments. In other instances, well-known materials or methods have not been described in detail in order to avoid obscuring the present embodiments.

Reference throughout this specification to “one embodiment”, “an embodiment”, “one example” or “an example” means that a particular feature, structure or characteristic described in connection with the embodiment or example is included in at least one embodiment of the present embodiments. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus. Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.”

### Industrial Applicability

The claimed invention has industrial applicability in the biomedical arts. In particular, the claimed invention is directly relevant to the therapeutic administration of pharmaceuticals for mitigation of and therapeutic effects against Alzheimer's disease as well as managing proactive lifestyle changes.

Sequence Listing

Seq. ID. No. 1
miR-4508
GCGGGGCUGGGCGCGCG
Seq. ID. No. 2
miR-6087
UGAGGCGGGGGGGCGAGC
Seq. ID. No. 3
miR-133a-3p
UUUGGUCCCCUUCAACCAGCUG
Seq. ID. No. 4
miR-1-3p
uggaauguaaagaaguauguau
Seq. ID. No. 5
miR-4492
GGGGCUGGGCGCGCGCC

I claim:

1. A personal health Alzheimer's monitoring system comprising:
A saliva sample collection device, a smartphone personal communication device incorporating one or more central processing units, one or more cameras, internet connection means, health sample interpretation software, artificial intelligence element and cloud computing element with real-time interpretation and communication of saliva sample health care real-time data in conjunction with results received from health sample subsequent analysis hardware.

2. The system of claim 1 wherein said saliva sample collection device additionally comprises one or more health sample detection chemicals.

3. The saliva sample collection device of claim 2 wherein said one or more saliva sample detection chemicals additionally comprises a compound having the following structural formula:

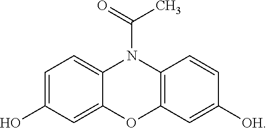 [SMILES: CC(=O)N1c2ccc(O)cc2Oc2cc(O)ccc21]

4. The system of claim 3 wherein said health sample subsequent analysis hardware results additionally comprises genetic analysis hardware reporting microRNA levels corresponding to Sequence ID #1.

5. The system of claim 4 wherein said health sample subsequent analysis hardware results additionally comprises genetic analysis hardware reporting microRNA levels corresponding to Sequence ID #2.

6. The system of claim 5 wherein said health sample subsequent analysis hardware results additionally comprises genetic analysis hardware reporting microRNA levels corresponding to Sequence ID #3.

7. The system of claim 6 wherein said health sample subsequent analysis hardware results additionally comprises genetic analysis hardware reporting microRNA levels corresponding to Sequence ID #4.

8. The system of claim 7 wherein said health sample subsequent analysis hardware results additionally comprises genetic analysis hardware reporting microRNA levels corresponding to Sequence ID #5.

9. The system of claim 8 wherein said health sample subsequent analysis hardware additionally comprises enzyme-linked immunosorbent assay (ELISA) chemical analysis functionality.

10. The system of claim 9 wherein said health sample subsequent analysis hardware additionally comprises chromatography and mass spectrometry functionality.

11. The system of claim 10 wherein said health sample analysis subsequent hardware additionally comprises sample fungal analysis.

12. The system of claim 11 wherein said health sample analysis subsequent hardware additionally comprises sample microbial analysis.

13. The system of claim 12 wherein said health sample analysis subsequent hardware additionally comprises sample viral analysis.

14. A method for personal health data monitoring comprising the steps of:
Sample preparation by exposing a sample collection means to saliva,
Real-time glucose data capture by smartphone optical acquisition,
Subsequent sample genetic analysis,
Data transmission wherein user microRNA results are sent to a user's smartphone device,
Data reporting wherein a user's microRNA levels are presented in the form of Alzheimer's risk factor assessment.

15. The method for personal Alzheimer's monitoring of claim 14 additionally comprising a data alert wherein abnormal microRNA levels are reported to the user's family members and designated medical providers.

16. The method for personal health data monitoring of claim 14 wherein said microRNA levels are selected from the group consisting of microRNA 4508, microRNA-6087, microRNA-133a-3P, microRNA-1-3p and microRNA-4492.

17. The method for personal health data monitoring of claim 16 additionally comprising:
Further subsequently analyzing said saliva sample during said sample data analysis for Alzheimer's risk factor indicators utilizing liquid chromatography, mass spectrometry and genetic sequencing techniques to identify fungal, bacterial and viral Alzheimer's risk indicators.

18. A personal health Alzheimer's monitoring system comprising:
A non-nitrocellulose saliva sample collection lateral flow strip, a smartphone personal communication device incorporating one or more central processing units, one or more cameras, internet connection means, health sample interpretation software, artificial intelligence element and cloud computing element with real-time interpretation and communication of saliva sample health care real-time data in conjunction with results received from health sample subsequent analysis hardware. microRNA levels are selected from the group consisting of microRNA 4508, microRNA-6087, microRNA-133a-3P, microRNA-1-3p and microRNA-4492.
